(19) United States
(12) Patent Application Publication
(10) Pub. No.: US 2015/0069271 A1
(43) Pub. Date: Mar. 12, 2015

(54) SYSTEM FOR STERILISING PLASTIC PARISONS WITH SIMULTANEOUS INTERNAL AND EXTERNAL STERILIZATION

(71) Applicant: KRONES AG, Neutraubling (DE)

(72) Inventors: Juergen Soellner, Beratzhausen (DE); Hans Scheuren, Bad Kreuznach (DE); Holger Mueller, Pentling (DE)

(21) Appl. No.: 14/474,974

(22) Filed: Sep. 2, 2014

(30) Foreign Application Priority Data
Sep. 11, 2013 (DE) 10 2013 109 988.2

(51) Int. Cl. A61L 2/08; A61L 2/24; B65G 29/00

(52) U.S. Cl. CPC A61L 2/087; B65G 29/00; A61L 2/24; A61L 2202/23
USPC 250/492.3

(57) ABSTRACT

A system for sterilizing plastic containers and in particular plastic parisons with a transport device which transports the plastic parisons along a predetermined transport path (P), has a first sterilizing device which is suitable for applying radiation to an external surface of the plastic containers in order to sterilise this external surface, and a second sterilizing device which is suitable for applying radiation to an internal surface of the plastic containers in order to sterilise this internal surface. At least one holding device is disposed on the transport device and has an engaging element which can be introduced into a mouth region of the plastic container in order to hold it, wherein at least one component of the second sterilizing device is integrated into this holding device.

The present invention relates to a system and a method for sterilising plastic containers and in particular plastic parisons. It is known from the prior art that plastic containers or containers in general are sterilised before they are filled. For this sterilisation several methods are known, such as for example sterilisation by means of hydrogen peroxide. Recently, however, attempts have been made to dispense with the use of such chemicals. Therefore there have been moves to change over to carrying out the sterilisation by irradiation and in particular, but not exclusively, by irradiation with charge carriers such as for example electrons.

In the prior art plastic parisons run into a blow moulding machine after heating or after an oven. From internal prior art in the name of the applicant it is known that after the oven the plastic parisons run into a sterilisation module via a feed starwheel, then are externally disinfected by stationary emitters and then are guided via a transport device to an internal disinfection module in which fingers protrude into the plastic parisons and internally sterilise them. Then the plastic parisons are transported onwards by means of a discharge starwheel. During the external disinfection the plastic parisons are held on their inner face by means of a gripping element.

However in this way the disinfection of the plastic parisons is relatively complex and also presupposes a plurality of modules disposed one after the other. The object of the present invention therefore is to provide a system and a method which reduce the cost of sterilisation of plastic parisons. This object is achieved according to the invention by the subject matter of the independent claims. Advantageous embodiments and modifications are the subject of the subordinate claims.

A system according to the invention for sterilising plastic containers and in particular plastic parisons has a transport device which transports the plastic parisons along a predefined transport path. Furthermore the system has a first sterilising device which is suitable for applying radiation to an external surface of the plastic containers in order to sterilise this external surface. Furthermore the system has a second sterilising device which is suitable for applying radiation to an internal surface of the plastic containers in order to sterilise this internal surface.

According to the invention at least one holding device is disposed on the transport device and has an engaging element which can be introduced into a mouth region of the plastic container in order to hold it. In this case at least one component of the second sterilising arrangement is integrated into this holding device. It is also proposed that a holding device is provided which in addition to the actual holding function also has a sterilising function. Therefore a sterilising element is preferably used which is already integrated into a holding device and in particular an internal gripping device for the plastic parisons. The holding device is preferably an internal gripper.

The radiation is in particular particle radiation or charge carrier radiation and particularly preferably electron radiation. However, other types of radiation or charge carriers could also be used, such as for example protons, alpha particles and the like.

In a further advantageous embodiment the system has an electron generating device and preferably also an accelerating device which accelerates the charge carriers or the electrons in a predetermined acceleration direction.

In a further advantageous embodiment the system also has an exit window through which the charge carriers can exit and thus for example can reach an inner wall of the containers.

In a further advantageous embodiment the system has a sterile chamber inside which the plastic parisons are transported. Furthermore a sealing means is preferably provided which seals this sterile chamber relative to the surroundings.

This sterile chamber is advantageously constructed like a channel around a transport path of the plastic parisons. The system advantageously also has sealing means which seal the sterile chamber and in particular sealing means which seal walls which are movable with respect to one another. Thus in a possible embodiment as sealing means a so-called water lock can be used which has a circumferential water-filled channel into which a blade of the relatively movable component protrudes.

In a further advantageous embodiment the transport device transports the plastic containers at least in some sections along a circular path. In a further advantageous embodiment the transport device transports the plastic containers at least during the internal sterilisation thereof at least in some sections along a circular path.

In a further advantageous embodiment a delivery device is provided which delivers the plastic parisons to the transport device. The transport device advantageously has a plurality of holding devices for holding the plastic containers.

Furthermore moving devices are advantageously also provided which enable a relative movement between the plastic containers and the holding devices in a longitudinal direction of the plastic containers. In this case the holding device are advantageously moved relative to the plastic containers. These moving devices may for example be electric motors and in particular linear motors, which are associated with the individual holding devices, so that an individual control of the movements of the individual holding devices is possible. In addition. however, it would also be possible to produce the movement of the holding devices by means of guide curves.

In a further advantageous embodiment the first sterilising device is disposed along the transport path of the holding device in such a way that the plastic containers are at least intermittently sterilised simultaneously by the first sterilising device and by the second sterilising device. Therefore in contrast to the prior art the system can function with only one module for carrying out an internal and external sterilisation.

In other words the external and internal sterilisation or external and internal disinfection is carried out simultaneously in order to give the module a more compact configuration. This is achieved in that the second sterilising device is integrated into the holding device, for example the preform internal gripper, which is otherwise a component of the external sterilisation.

In the course of the method it is conceivable that the plastic parisons are for example transported laterally in their thread or support ring for instance by a feed starwheel. On the transfer path just before the entry into the external disinfection the finger with the integrated emitter preferably already accompanies the plastic parison. This means that the holding device is located above the plastic containers and can thus already disinfect the internal surface of the plastic containers or plastic parisons.

During transport through the external disinfection the holding device then moves into the plastic parison and holds it. During this time irradiation of both the external surface and also the lower internal surface of the plastic parisons takes place. In this time period the upper internal surface cannot be reached because of the internal gripper or the holding device. However, at the discharge from the external disinfection the holding device moves out of the plastic parison again (as soon as the holding device on the discharge starwheel holds the plastic parison from the exterior) and again accompanies the plastic parison, so that again the entire (upper and lower) internal surface of the plastic parison is disinfected.

Advantageously, therefore, during the sterilisation of the lower region of the plastic parison the holding device does not move relative to the plastic parison, but the holding device is preferably introduced only to a certain extent, in particular into a mouth region of the plastic parison.

The path which the plastic container travels during sterilisation can also be shortened by this procedure. In the prior art because of the relatively long travel of the plastic parisons through the modulus and the internal and external disinfection which does not take place simultaneously there is a potential for recontamination of the plastic parisons which have already been treated. Furthermore the plastic parisons cool due to the comparatively long travel, wherein on the other hand a specific time must not be exceeded, since otherwise the temperature profile applied by the previously set oven is smudged? and the plastic parisons cool again and thus blow moulding will be poor or may not occur at all.

In a further advantageous embodiment the system has a delivery device which delivers the plastic containers to the holding device and this delivery device or the transport device are designed in such a way that they can change a spacing between two adjacent plastic containers along the transport path. The delivery device is advantageously designed in this way. Therefore it is proposed in particular to carry out the delivery by means of a so-called delayed division star which can delay the division between the individual plastic parisons.

Advantageously the delivery device also does not transport the plastic parisons in this region along a precisely circular path, but it is conceivable that the plastic parisons in the transfer zone are moved along a specific portion of the path with the holding devices of the sterilising device. In other words, in order to compensate for the treatment shadow caused by the end gripper, the transfer into and/or out of the treatment carousel is prolonged.

The above-mentioned pitch reduction starwheel which transfers the plastic containers to the treatment carousel or the holding means thereof accompany the plastic containers and the holding device during the transfer or the end gripper remains at the top under cam control until the last moment or spaced apart from the mouth of the plastic container. In this time period the second sterilising device can already radiate permanently and thus disinfect the internal mouth region of the plastic parisons. During movement out of the treatment carousel the plastic parisons are immediately transferred to a further pitch reduction starwheel, the holding device moves upwards and the mouth region now freed again is treated for a second time by the accompanying of the pitch reduction starwheel on the treatment carousel.

By this procedure the volume of the entire module may become substantially smaller and thus also necessitates less expenditure on radiation protection, which leads to a saving of material. Furthermore, as mentioned above, the risk of recontamination is also reduced. In this way the entire module can be reduced by two transport starwheels and thus the mechanically costly transfers are also reduced and the entire system becomes more stable in operation of the process. Due to the shorter transit time through the module the problem of the equalisation time also vanishes (the plastic parison must be blow moulded a short time after the heating). Furthermore due to the simpler structure of the module the targeted management of the necessary ventilation technology can be made simpler. Less sterile air is required in order to maintain an overpressure relative to the surroundings.

Thus in a further embodiment the transport device and/or the delivery device transports the plastic containers along portions of the transport path which have different radii of curvature. The same procedure is also conceivable in the region of the discharge unit from the transport device.

In a further advantageous embodiment the delivery device has at least one second holding device which is suitable for gripping the plastic container on an external region. In particular this may involve gripping clamps which grip the plastic containers below a support ring, in particular controllable clamps.

Furthermore the present invention is directed to a holding device for holding plastic containers. This holding device has an engaging element which can be introduced into a mouth region of the plastic container in order to hold it.

According to the invention a sterilising device is integrated into this holding device, wherein this sterilising device has an exit window through which charge carriers can exit in order to sterilise an inner wall region of the plastic containers. Thus within the context of the invention a special holding device, in particular a holding mandrel, is also proposed, which in addition to the holding function also has the possibility of carrying out a sterilisation, in particular of an inner wall of a plastic containers and in particular of a plastic parison. In the prior art such sterilising devices are usually introduced in the manner of a beam finger into the container, that is to say for example that they are moved relative to the plastic container during the sterilisation.

In the proposed variant the holding device is preferably not moved at least intermittently relative to the plastic parison but holds the plastic parison in particular at its mouth region. This is suitable in particular in the sterilisation of plastic parisons.

In a further advantageous embodiment the holding device also has a cooling device in order to cool the exit window. In this case it is provided in particular that an air stream is delivered to the exit window. Sterile air is advantageously delivered to the exit window. Thus for example cooling air channels can be provided which also extend in a longitudinal direction of the holding device. The holding device advantageously has a housing or a closed compartment within which the electrons move in order finally to exit through the exit window. A vacuum or a partial vacuum is advantageously formed inside this housing. In addition the individual sterilising devices preferably also each have charge carrier generating arrangements and preferably also have accelerating devices which accelerate the generated charge carriers, in particular electrons, in the direction of the exit window.

In addition it would also be conceivable and preferable that the radiation devices or finger emitters guide the (cooling) air through a double wall. Thus for example a further tube, preferably a titanium coupling tube, can be disposed around the housing of the beam finger. Between these tubes the cooling air can reach the exit window and cool it.

In addition. however, it would also be possible to guide the cooling air at least in some sections over the holding devices. These holding devices may for example have channels for conveying the cooling air. Also the cooling air could be guided between components of the holding device, for instance between two adjacent projections of the holding device. Additionally or alternatively it would also be conceivable to use a fluid cooling system.

The cooling device is advantageously constructed in such a way that it supplies the exit window from outside with the coolant, for example with air. However, other cooling devices, such as for example fluid cooling systems, would also be conceivable (additionally or alternatively).

In a further advantageous embodiment the said housing, within which the charge carriers reach the exit window, is surrounded at least in some sections by the engaging element. In this way a very compact design of the holding device is conceivable.

In an advantageous embodiment the engaging element has a plurality of engaging portions which can be clamped onto the mouth. In this case between the individual engaging portions gaps can be provided which also extend in a longitudinal direction of the holding device.

In a further advantageous embodiment the exit window projects in a longitudinal direction of the holding device relative to the engaging element. This means that the exit window projects in the direction of the plastic parison or of the base region thereof relative to the engaging element. In this way the exiting charge carriers are not hindered by regions of the engaging element in the sterilisation of the plastic parisons. Thus sterilisation can take place in all circumferential directions.

In a further preferred embodiment the system has a vacuum generating device which supplies the plastic container at least intermittently with a negative pressure during sterilisation. This procedure is suitable in particular for the sterilisation of plastic parisons, since these have a relatively high wall thickness and therefore can be supplied with a vacuum without deforming.

In addition the vacuum supply device could be constructed in such a way that it sucks air out of the plastic container and this air is then at least partially used for cooling the exit window. Thus it would be conceivable to use a specific proportion of the extracted air again for cooling of the (same) exit window. In addition the air which is extracted from a specific plastic parison could be used for cooling a further plastic parison.

Furthermore the present invention is directed to a method for sterilising plastic containers and in particular plastic parisons. In this case the plastic parisons are transported by means of a transport device along a predetermined transport path and during this transport at least one external surface of the plastic parisons is supplied by a first sterilising device with radiation which sterilises these external surfaces.

According to the invention the plastic containers are transported by means of holding devices which are introduced into the plastic containers in a mouth region thereof, wherein at least intermittently (in particular during the transport of the plastic parisons by means of the transport device) an internal surface of the plastic containers is supplied by a second sterilising device with radiation which sterilises this internal surface and at least intermittently the supply to the internal surface and the supply to the external surface take place simultaneously.

The radiation is advantageously charge carrier radiation and in particular electron radiation. Thus at least intermittently the containers are irradiated both from the interior and also from the exterior.

As mentioned above, a sterilising device is advantageously integrated into the holding device, wherein this radiation device or the sterilising device acts on the internal surface of the plastic containers from the interior.

In a further advantageous method the containers are delivered by means of a delivery device to the transport device. The plastic containers are advantageously delivered along a transport path which is to some extent circular but to some extent also non-circular. Thus in particular in a transfer zone it may be provided that the plastic containers are for example conveyed in a straight line or along changing radii of curvature.

In a further advantageous embodiment the delivery device grips the plastic containers from the exterior and the holding devices are also carried along at least intermittently during this transport. Also during the removal from the transport device the plastic parisons can be transported along or by means of different radii of curvature. Inner mouth regions of the plastic parison are advantageously sterilised chronologically before and/or after the sterilisation of the external regions.

In a further advantageous method the plastic containers are at least intermittently transported during sterilisation inside a clean chamber.

In a further advantageous method, during a feeding movement of the holding device towards the plastic containers and/or during a movement of the holding device away from the plastic containers sterilisation of the internal mouth region and in particular of substantially the entire internal mouth region of the plastic containers and/or an upper mouth region is carried out.

The upper mouth region is understood be in particular the portion which is directly adjacent to the rim of the mouth of the plastic container. This is preferably a portion which is located in the region of an (external) thread of the plastic container and/or a region between the external thread and the rim of a mouth (relative to a longitudinal direction of the plastic container which extends from the mouth of the container to a base, in particular a base cone).

In a further advantageous embodiment the system for sterilising the plastic containers is disposed after a heating device and/or before the end of a transforming machine. This transforming machine may in particular be a blow moulding machine and in particular a stretch blow moulding machine or the blow wheel thereof.

In this case sterilisation of this internal mouth region is advantageously carried out by means of radiation and in particular by means of charge carrier radiation. During the movement forward and/or back the upper external surfaces of the plastic containers are preferably sterilised. Thus in particular the thread of the plastic container or plastic parison can be sterilised. In a further advantageous method the holding devices are advanced towards the plastic containers in the longitudinal direction of the plastic containers. Sterilisation of the direct internal mouth regions of the plastic parisons advantageously takes place during delivery to the transport device and/or during removal from the transport device.

In the sterilisation of the plastic container or plastic parison the following steps are preferably carried out, wherein it is pointed out that several of the said steps can be omitted:
The plastic container is guided under the finger beam or the (second) sterilising arrangement. The plastic container is disinfected in the mouth region;
The holding device moves into the plastic container;
The second sterilising arrangement, in particular a beam finger moves further into the plastic container (which however takes place in particular through a movement of the plastic containers); in this step charge carriers or electrons can preferably already be emitted by the sterilising device in order to sterilise the inner wall of the plastic container. Preferably, however, as stated, the beam finger is not moved here but conversely the plastic container with the holding device is moved towards the beam finger;
The second sterilising arrangement, in particular a beam finger, moves into a base region of the plastic container; in this step charge carriers or electrons can preferably already be emitted by the sterilising device in order to sterilise the inner wall of the plastic container.
The plastic container and the holding device are moved downwards, and thus the sterilising device is removed from the plastic container. Then the plastic container is transferred to an external gripper and the disinfected preform is moved on; also during this withdrawal of the sterilising device or the movement downwards of the plastic container sterilisation of the (in particular external) mouth region of the plastic container can again take place.

Further advantages and embodiments are apparent from the appended drawings. In the drawings:

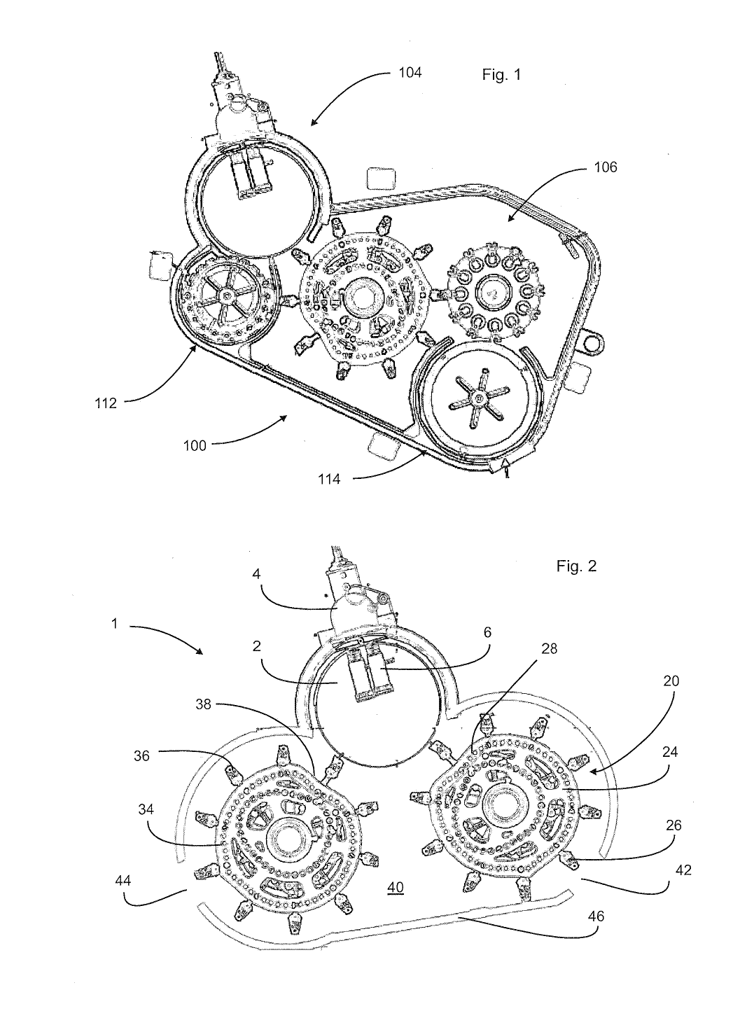

FIG. 1 shows a sterilising arrangement according to the internal prior art in the name of the applicant;

FIG. 2 shows a schematic representation of a sterilising device according to the invention;

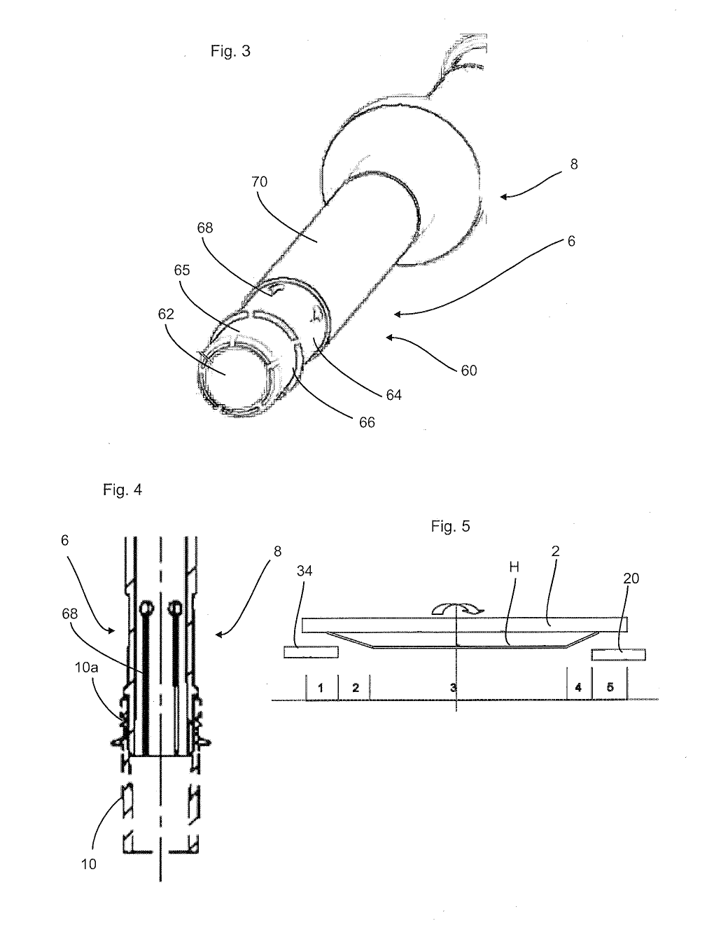

FIG. 3 shows a schematic representation of a holding device with integrated sterilising device;

FIG. 4 shows a sectional representation of a sterilising device introduced into a plastic parison; and

FIG. 5 shows a container sterilisation sequence.

FIG. 1 shows a system 100 for sterilising containers according to the internal prior art in the name of the applicant. In this first of all a delivery device 112 is provided which delivers the plastic parisons to an external sterilisation unit 104. Starting from this external sterilisation unit 104 the plastic containers are removed and guided to an internal sterilisation unit 106. Finally the plastic containers are discharged again by means of a discharge unit 114. In this way in all two delivery units 112, 114 as well as a further transfer unit are necessary between the two sterilising devices 104 and 106.

FIG. 2 shows a schematic representation of a system 1 according to the invention for sterilising plastic parisons. In this case the plastic parisons are first of all delivered via a delivery opening 44 to a delivery device 34. This delivery device 34 has a plurality of holding devices which hold the plastic parisons for example on the external circumference thereof, for example below the support ring thereof. Furthermore the delivery device 34 has a transfer zone 38 in which the holding devices and thus also the plastic parisons disposed thereon are preferably not guided along a circular transport path but by means of different radii of curvature, so that the transfer zone to the transport device 2 can be lengthened. The transport device 2 may be constructed here as a rotatable wheel.

The reference numeral 4 designates a first sterilising device which serves for external sterilisation of the plastic parisons. In this case this first sterilising device could be disposed for example stationary along a transport path of the plastic parisons.

The reference numeral 20 designates a discharge unit which serves for discharging the plastic parisons out of the system 1. This in turn also has a substrate 24 on which a plurality of holding devices 26 are arranged. These are in turn transported section by section (in the transfer zone 28) along a non-circular transport path.

The reference numeral 42 designates a further discharge opening via which the plastic parisons, now sterilised, are discharged from the system 1.

The reference numeral 40 designates a clean chamber within which the sterilisation and also the transport of the plastic parisons take place. This clean chamber is sealed by means of walls 46 relative to (unsterile) surroundings. Sterilising devices and/or cleaning devices which act on components of the system, such as for instance the holding devices 36, 26, for cleaning and/or sterilisation with a free-flowing sterilising and/or cleaning agent (for example hydrogen peroxide or peracetic acid), could also be disposed within this clean chamber. In this way a so-called CP cleaning of the installation could also be enabled. However, sterilisation of system parts by means of electromagnetic and/or electron radiation could take place.

FIG. 3 shows an embodiment of a second sterilising device which is integrated into a holding device 8. In this case the reference numeral 70 identifies a main body of this holding device and the reference numeral 64 identifies a plurality of holding elements or engagement portions which can rest outwardly against an inner wall of the plastic parisons in order to hold it. The reference numeral 68 identifies slots which are disposed between the individual holding elements. The reference numeral 65 identifies a second housing portion within which the electrons are movable. The reference numeral 62 identifies the exit window by means of which the charge carriers or electrons can exit.

FIG. 4 shows a representation in which the holding device 8 is used especially in a plastic parison 10. The reference numeral 10a identifies a mouth region of this plastic parison. Again the slots 68 are discernible between the individual holding elements.

FIG. 5 shows an illustration of the course of the sterilisation. In this case the delivery device 34 and the discharge unit 20 illustrated schematically. The reference numeral 2 in turn identifies the transport device which is also shown here in a developed view. The reference H designates a lifting curve of the holding device which holds the plastic parisons while they are being transported with the second transport device 2. It will be recognised that in a region 1 the holding device 36 of the delivery device 34 accompanies the holding device 8, since in this region the two schematically illustrated devices 34, 2 are shown overlapping. In this region 1a sterilisation of the mouth region of the plastic parison can take place, in particular by the second sterilising arrangement.

In the region 2 the holding device 8 or the end gripper moves into the plastic parison and the holding device 36 of the delivery device 34 is removed. In this time period an internal sterilisation of a lower region of the plastic parison can also already take place.

In the region 3 an external treatment of the plastic parison by the first sterilising arrangement also takes place. In addition in this region an internal sterilisation of the lower region of the plastic parison can also take place.

In the region 4 the holding device of the discharge unit 20 grips the plastic parison and the holding device 8 is removed upwards, as illustrated by the lifting curve H. Also in this region the plastic parison is treated for a second time in its mouth region, in particular in the upper mouth region.

In the region 5, similar as in the region 1, both the holding device of the discharge unit and also the holding device 8 of the transport device 2 accompany the plastic parison. Thus here too a mouth region of the plastic parison is sterilised for a second time.

As stated, the delivery device 34 and/or the discharge unit 20 are preferably each configured as so-called pitch reduction starwheels. In this case it is possible that the individual holding elements of this delivery device and/or of the discharge unit are movable relative to a rotatable carrier, in particular movable in a transport plane of the plastic containers. Thus the individual holding devices 36, 26 could for example be pivotable relative to the rotatable carrier and/or also linearly movable.

The applicant reserves the right to claim all the features disclosed in the application documents as essential to the invention in so far as they are individually or in combination novel over the prior art.

### LIST OF REFERENCE SIGNS

1 system
2 transport device
4 first sterilising device
6 second sterilising device
10 plastic parison
10a mouth region
20 discharge unit
24 carrier
26 holding devices
28 transfer zone
34 delivery device
38 transfer zone
40 clean chamber
42 discharge opening
44 delivery opening
46 walls
62 exit window
64 holding elements
65 second housing section
68 slots
70 main body
100 system for sterilising containers
104 external sterilisation unit
106 internal sterilisation unit
112 delivery device
114 discharge unit

1. A system for sterilising plastic containers and in particular plastic parisons with a transport device which transports the plastic parisons along a predetermined transport path, including a first sterilising device which is suitable for applying radiation to an external surface of the plastic containers in order to sterilise this external surface, and a second sterilising device which is suitable for applying radiation to an internal surface of the plastic containers in order to sterilise this internal surface, wherein at least one holding device is disposed on the transport device and has an engaging element which can be introduced into a mouth region of the plastic container in order to hold it, wherein at least one component of the second sterilising device is integrated into the holding device.

2. The system according to claim 1, wherein the first sterilising device is disposed along a transport path of the holding device in such a way that the plastic containers are at least intermittently sterilised simultaneously by the first sterilising device and by the second sterilising device.

3. The system according to claim 1, wherein the system has a delivery device which delivers the plastic containers to the holding device and this delivery device or the transport device are designed in such a way that they can change a spacing between two adjacent plastic containers along the transport path.

4. The system according to claim 1, wherein the transport device and/or the delivery device and/or a discharge unit transports the plastic containers along portions of the transport path which have different radii of curvature.

5. The system according to claim 3, wherein the delivery device has at least one second holding device which is suitable for gripping the plastic containers on an external region.

6. A holding device for holding plastic containers with an engaging element which can be introduced into a mouth region of the plastic container in order to hold it, wherein a sterilising device is integrated into this holding device, wherein this sterilising device has an exit window through which charge carriers can exit in order to sterilise an inner wall region of the plastic containers.

7. The holding device according to claim 6, wherein the sterilising device has a housing inside which charge carriers reach the exit window.

8. The holding device according to claim 7, wherein the housing is surrounded at least in some sections by the engaging element.

9. The holding device according to claim 6, wherein the exit window projects in a longitudinal direction (L) of the holding device relative to the engaging element.

10. A method for sterilising plastic containers and in particular plastic parisons, wherein the plastic containers are transported by a transport device along a predetermined transport path, and during this transport at least intermittently an external surface of the plastic containers is supplied by a first sterilising device with radiation which sterilises this external surface, wherein the plastic containers are transported by holding devices which are introduced into plastic containers in a mouth region thereof, wherein at least intermittently during the transport of the plastic parisons by the transport device an internal surface of the plastic containers is supplied by a second sterilising device with radiation which sterilises this internal surface and at least intermittently the supply to the internal surface and the supply to the external surface take place simultaneously.

11. The method according to claim 10, wherein during a feeding movement of the holding device towards the plastic containers and/or during a movement of the holding device away from the plastic containers sterilisation of the internal mouth region and in particular of an upper mouth region of the plastic containers is carried out.

12. A system according to claim 1, wherein the system for sterilising the plastic containers is disposed after a heating device and/or before the end of a transforming machine.

13. The system according to claim 2, wherein the system has a delivery device which delivers the plastic containers to the holding device and this delivery device or the transport device are designed in such a way that they can change a spacing between two adjacent plastic containers along the transport path.

14. The system according to claim 2, wherein the transport device and/or the delivery device and/or a discharge unit transports the plastic containers along portions of the transport path which have different radii of curvature.

15. The system according to claim 13, wherein the delivery device has at least one second holding device which is suitable for gripping the plastic containers on an external region.

16. The system according to claim 3, wherein the transport device and/or the delivery device and/or a discharge unit transports the plastic containers along portions of the transport path which have different radii of curvature.

17. The system according to claim 16, wherein the delivery device has at least one second holding device which is suitable for gripping the plastic containers on an external region.
